(19) United States
(12) Patent Application Publication
(10) Pub. No.: US 2019/0269687 A1
(43) Pub. Date: Sep. 5, 2019

(54) In Vivo Chemoselection with Low Dose Thioguanine

(71) Applicant: Calimmune, Inc., Tucson, AZ (US)

(72) Inventors: Jeffrey S. Bartlett, Columbus, OH (US); Louis Randall Breton, Tucson, AZ (US)

(21) Appl. No.: 16/411,830

(22) Filed: May 14, 2019

Related U.S. Application Data
(63) Continuation of application No. PCT/US17/62127, filed on Nov. 17, 2017.
(60) Provisional application No. 62/423,794, filed on Nov. 18, 2016.

(51) Int. Cl. A61K 31/52; A61P 37/02

(52) U.S. Cl. CPC A61K 31/52; A61P 37/02

(57) ABSTRACT

The present disclosure is directed to methods of selecting for modified stem cells in vivo. The present disclosure is also directed to a 6TG dosing schedule. The present disclosure is also directed to an oral formulation comprising 6TG.

### CROSS-REFERENCE TO RELATED APPLICATIONS

This application is a continuation of International Application No. PCT/US17/62127 filed on Nov. 17, 2017, which claims priority to and the benefit of the filing date of U.S. Provisional Patent Application No. 62/423,794 filed on Nov. 18, 2016, the disclosures of which are hereby incorporated by reference herein in their entireties.

### FIELD OF DISCLOSURE

This disclosure generally relates to the fields of pharmaceuticals and biotechnology. The disclosure relates to conferring myeloprotection by dosing with certain active pharmaceutical ingredients. The disclosure also relates to dosing with active pharmaceutical ingredients prior to stem cell engraftment.

### STATEMENT OF INDUSTRIAL APPLICABILITY

The present disclosure has industrial applicability in the field of medicine and gene therapeutics.

### BACKGROUND

6-thioguanine (6-TG), introduced into the clinic in the early 1950s, is a well-studied purine analog having both anticancer and immune-suppressive activities. Thioguanine competes with hypoxanthine and guanine for the enzyme hypoxanthine-guanine phosphoribosyltransferase (HGPRTase) and is itself converted to 6-thioguanylic acid (TGMP). This nucleotide reaches high intracellular concentrations at therapeutic doses. TGMP interferes several points with the synthesis of guanine nucleotides. It inhibits de novo purine biosynthesis by pseudo-feedback inhibition of glutamine-5-phosphoribosylpyrophosphateamidotransferase—the first enzyme unique to the de novo pathway for purine ribonucleotide. TGMP also inhibits the conversion of inosinic acid (IMP) to xanthylic acid (XMP) by competition for the enzyme IMP dehydrogenase. At one time TGMP was felt to be a significant inhibitor of ATP:GMP phosphotransferase (guanylate kinase), but recent results have shown this not to be so. Thioguanylic acid is further converted to the di- and tri-phosphates, thioguanosine diphosphate (TGDP) and thioguanosine triphosphate (TGTP) (as well as their 2′-deoxyribosyl analogues) by the same enzymes which metabolize guanine nucleotides.

### BRIEF SUMMARY OF THE DISCLOSURE

In one aspect of the present disclosure is a method of increasing stem cell engraftment following a hematopoietic stem cell transplantation comprising administering to a human patient a dose of a thiopurine. In some embodiments, the thiopurine is 6TG. In some embodiments, the total dose of the thiopurine within any administration cycle does not exceed 7 mg/kg.

In another aspect of the present disclosure is a method of increasing stem cell engraftment following a hematopoietic stem cell transplantation comprising administering to a human patient a dosage of 6TG ranging from between about 0.2 mg/kg/day to about 0.6 mg/kg/day. In some embodiments, the total dose of 6TG within any administration cycle does not exceed 7 mg/kg. In some embodiments, the administration cycle comprises between 3 and 15 doses.

In another aspect of the present disclosure is a method of selectively depleting HPRT wild-type cells comprising administering to a human patient a dosage of 6TG ranging from between about 0.2 mg/kg/day to about 0.6 mg/kg/day following a stem cell transplant. In some embodiments, the dosage ranges from between about 0.3 mg/kg/day to about 0.5 mg/kg/day. In some embodiments, the dosage is about 0.4 mg/kg/day. In some embodiments, the dosage is administered to the human patient one to three times a week on a schedule with a cycle selected from the group consisting of: (i) weekly; (ii) every other week; (iii) one week of therapy followed by two, three or four weeks off; (iv) two weeks of therapy followed by one, two, three or four weeks off; (v) three weeks of therapy followed by one, two, three, four or five weeks off; (vi) four weeks of therapy followed by one, two, three, four or five weeks off; (vii) five weeks of therapy followed by one, two, three, four or five weeks off; and (viii) monthly.

In some embodiments, between about 3 and about 10 dosages of 6TG are administered to the patient over an administration period ranging from 1 week to about 4 weeks. In some embodiments, 4 or 5 dosages of 6 TG are administered to the patient over a 14-day period. In some embodiments, the dosages are spaced apart over equal time periods. In some embodiments, subsequent dosages are made every third day. In some embodiments, each dosage comprises the same amount of 6TG. In some embodiments, at least one subsequent dose comprises an amount of 6TG greater than an amount of 6TG in an initial dosage. In some embodiments, the cycle is repeated 4, 6, 8, or 10 times. In some embodiments, a time period between dosing with 6TG and the stem cell transplant ranges from between about 2 weeks to about 6 weeks. In some embodiments, the time period ranges from between about 3 weeks to about 4 weeks.

In some embodiments, subsequent doses of 6TG are separated by a period of at least one day. In some embodiments, an amount of the 6TG administered is based on a measured HPRT-enzyme activity level. In some embodiments, a total amount of 6TG administered does not exceed 5 mg/kg.

In another aspect of the present disclosure is a method of conferring myeloprotection comprising: (i) performing a myleosuppressive conditioning step prior to a stem cell transplantation, the myelosuppressive conditioning step comprising administering 6TG in an amount effective to induce selective myelotoxicity; and (ii) performing a post-transplantation chemoselection step following stem cell transplantation, wherein the post-transplantation conditioning step comprises administering one or more doses of 6TG, where each of the one or more doses comprises an amount of 6TG ranging from about 0.3 mg/kg to about 0.5 mg/kg. In some embodiments, the post-transplantation chemoselection step comprises administering 6TG over one or more administration cycles. In some embodiments, the total dose of 6TG in any single administration cycle does not exceed 6 mg/kg. In some embodiments, the total dose does not exceed 5 mg/kg.

In some embodiments, the amount of 6TG for post-transplantation conditioning is about 0.4 mg/kg. In some embodiments, between about 3 and about 10 doses of 6TG are administered post-transplantation to the patient over an administration period ranging from 1 week to about 4 weeks. In some embodiments, or 5 doses of 6TG are administered post-transplantation to the patient over a 14-day period. In some embodiments, post-transplantation doses are spaced apart over equal time periods. In some embodiments, subsequent post-transplantation doses are made every third day. In some embodiments, each post-transplantation dosage comprises the same amount of 6TG. In some embodiments, at least one subsequent post-transplantation dose comprises an amount of 6TG greater than an amount of 6TG in an initial dose. In some embodiments, the cycle is repeated 4, 6, 8, or 10 times. In some embodiments, an amount of 6TG administered per dose in a myleosuppressive conditioning step ranges from about 0.8 mg/kg to about 3 mg/kg. In some embodiments, multiple doses are provided in the myleosuppressive conditioning step, e.g. from 2 to about 15 doses, where each dose comprises between about 0.8 mg/kg to about 3 mg/kg of 6TG. In some embodiments, similar dosage amounts and/or administration cycles are utilized for both the myleosuppressive conditioning step and the post-transplantation conditioning step. In some embodiments, the dosages used in the myleosuppressive conditioning step are greater than the dosages used in the post-transplantation conditioning step.

In another aspect of the present disclosure is a method of increasing stem cell engraftment comprising administering to a human patient one or more doses of 6TG, wherein an amount of 6TG in each dose ranges from between about 0.2 mg/kg/day to about 0.6 mg/kg/day following a stem cell transplant, wherein a total amount of 6TG administered in any administration cycle does not exceed 7 mg/kg. In some embodiments, the total amount of 6TG administered in the administration cycle does not exceed 6 mg/kg. In some embodiments, the total amount of 6TG administered in the administration cycle does not exceed 5 mg/kg. In some embodiments, the total amount of 6TG administered in the administration cycle does not exceed 4.5 mg/kg. In some embodiments, the amount of 6TG in each dose ranges from between about 0.3 mg/kg/day to about 0.5 mg/kg/day. In some embodiments, amount of 6TG in each dose is about 0.4 mg/kg/day. In some embodiments, about 3 and about 10 doses of 6TG are administered to the human patient over an administration period ranging from 1 week to about 4 weeks. In some embodiments, 4 or 5 doses of 6TG are administered to the patient over a 14-day administration period. In some embodiments, the dosages of 6TG are spaced apart over equal time periods. In some embodiments, the doses of 6TG are made every other day i.e. every two days. In some embodiments, the doses of 6TG are made every third day. In some embodiments, each dosage comprises the same amount of 6TG. In some embodiments, at least one subsequent dosage of 6TG comprises an amount of 6TG greater than an amount of 6TG in an initial dosage. In some embodiments, a time period between dosing the human patient with 6TG and the stem cell transplant ranges from between about 2 weeks to about 6 weeks. In some embodiments, the amount of the 6TG administered is based on a measured HPRT-enzyme activity level.

In another aspect of the present disclosure is a method of increasing stem cell engraftment following a hematopoietic stem cell transplantation comprising administering to a human patient a dosage of 6TG ranging from between about 0.2 mg/kg/day to about 0.6 mg/kg/day, but where a total cumulative dose of 6TG administered in any single administration cycle for chemoselection does not exceed 7 mg/kg.

In another aspect of the present disclosure is a formulation for the oral administration comprising 6TG, wherein the 6TG is present in an amount ranging from between about 0.2 mg to about 0.6 mg, and at least one pharmaceutically acceptable excipient.

In another aspect of the present disclosure is a formulation for oral administration comprising 6TG, wherein the 6TG is present in an amount ranging from between about 12 mg to about 20 mg, and at least one pharmaceutically acceptable excipient. In some embodiments, the at least one pharmaceutically acceptable excipient is selected from pregelatinized starch, croscarmellose sodium, povidone, lactose monohydrate, microcrystalline cellulose, and magnesium stearate, and combinations thereof. In some embodiments, the formulation comprises an immediate release portion comprising 6TG and an extended release portion comprising 6TG, and wherein the extended release portion permits release of 6TG over a time period ranging from between about 30 minutes to about 12 hours after administration. In some embodiments, an amount of the 6TG in the extended release portion ranges from about 50% to about 75% by weight of the extended release portion. In some embodiments, the extended release portion comprises (i) a wax, and (ii) a matrix-forming component selected from the group consisting of succinic acid, citric acid, malic acid, stearic acid, succinic acid, lactic acid, aspartic acid, glutamic acid, gluconic acid, acetic acid, formic acid, hydrochloric acid, sulphuric acid, phosphoric acid, hydrophilic polymers, polyethylene glycols, pH dependent acrylate polymers or copolymers, and pore forming agents.

Clinical efficacy using genetically modified hematopoietic stem cells remains dependent on imparting a selective advantage to the transplanted cells. Applicants have developed a method of selecting for such genetically modified hemapoietic stem cells in vivo. Without wishing to be bound by any particularly theory, it is believed that in vivo selection would allow dosing based on clinical need and outcomes; the therapeutic window could be dialed-in iteratively and pharmacologically based on clinical assessments. To some extent selection is based on myelotoxicity—at least in the HSC compartment. However, in animals at the doses that impact the HSC compartment, there is little to no toxicity of other hematopoietic compartments (lymphoid or myeloid). This selective toxicity toward HSC is an important aspect of the present disclosure and is based on HPRT levels in various cell types (HSC have high HPRT activity)—although could also be related to TPMT (detoxifies 6TG) levels, or overall levels of purine biosynthesis.

### DETAILED DESCRIPTION

The preset disclosure is directed to methods of selecting for modified stem cells in vivo. The present disclosure is also directed to a 6TG dosing schedule. The present disclosure is also directed to an oral formulation comprising 6TG.

As used herein, the singular terms “a,” “an,” and “the” include plural referents unless the context clearly indicates otherwise. Similarly, the word “or” is intended to include “and” unless the context clearly indicates otherwise.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein, the term “6TG” refers to 6-thioguanine and pharmaceutically acceptable salts, derivatives or analogs thereof.

As used herein, the terms “administer,” or “administration” as they apply to a subject or patient, a placebo subject, a research subject, an experimental subject, a cell, a tissue, an organ, or a biological fluid, refers, without limitation, to contact of an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods. Treatment of a cell encompasses contact of a reagent to the cell, as well as contact of a reagent to a fluid, where the fluid is in contact with the cell. “Administration” also encompasses in vitro and ex vivo treatments, e.g., of a cell, by a reagent, diagnostic, binding composition, or by another cell.

As used herein, the terms “hematopoietic cell transplant” or “hematopoietic cell transplantation” refer to bone marrow transplantation, peripheral blood stem cell transplantation, umbilical vein blood transplantation, or any other source of pluripotent hematopoietic stem cells. Likewise, the terms the terms “stem cell transplant,” or “transplant,” refer to a composition comprising stem cells that are in contact with (e.g. suspended in) a pharmaceutically acceptable carrier. Such compositions are capable of being administered to a subject through a catheter.

As used herein, “HPRT” is an enzyme involved in purine metabolism encoded by the HPRT1 gene. HPRT1 is located on the X chromosome, and thus is present in single copy in males. HPRT1 encodes the transferase that catalyzes the conversion of hypoxanthine to inosine monophosphate and guanine to guanosine monophosphate by transferring the 5-phosphorobosyl group from 5-phosphoribosyl 1-pyrophosphate to the purine. The enzyme functions primarily to salvage purines from degraded DNA for use in renewed purine synthesis (see FIG. 5).

As used herein, the terms “subject,” or “patient,” refers to a vertebrate animal, including a mammal. A human, homo sapiens, is considered a subject or patient.

In some embodiments, the present disclosure is directed to a method of selectively depleting cells, including stems cells, that express HPRT, i.e. HPRT wild-type cells. In some embodiments, the method comprises administering one or more doses of 6TG, where each of the one or more doses comprise an amount of the 6TG that is less than an amount of 6TG used in the treatment of cancer in a human patient. For example, the amount of 6TG in a dosage designed to treat cancer in a human patient may range from about 2 mg/kg to about 4 mg/kg.

In some embodiments, the method comprises administering one or more doses of 6TG, where each of the one or more doses comprise an amount of 6TG that is at least 5 times less than an amount of 6TG in a dosage designed to treat cancer. In other embodiments, the method comprises administering one or more doses of 6TG, where each of the one or more doses comprise an amount of 6TG that is at least 10 times less than the amount of 6TG in a dosage designed to treat cancer. In yet other embodiments, the method comprises administering one or more doses of 6TG, where each of the one or more doses comprise an amount of 6TG that is at least 15 times less than the amount of 6TG in a dosage designed to treat cancer. In further embodiments, the method comprises administering one or more doses of 6TG, where each of the one or more doses comprise an amount of 6TG that is at least 20 times less than the amount of 6TG in a dosage designed to treat cancer.

In some embodiments, an amount of 6TG administered per dosage to a patient for in vivo chemoselection ranges from about 0.05 to about 1 mg/kg/day. In some embodiments, an amount of 6TG administered per dosage to a patient for in vivo chemoselection ranges from about 0.05 to about 0.8 mg/kg/day. In some embodiments, an amount of 6TG administered per dosage to a patient for in vivo chemoselection ranges from about 0.05 to about 0.7 mg/kg/day. In other embodiments, the amount of 6TG administered for in vivo chemoselection ranges from about 0.1 to about 0.8 mg/kg/day. In other embodiments, the amount of 6TG administered for in vivo chemoselection ranges from about 0.1 to about 0.7 mg/kg/day. In other embodiments, the amount of 6TG administered for in vivo chemoselection ranges from about 0.2 to about 0.7 mg/kg/day. In other embodiments, the amount of 6TG administered for in vivo chemoselection ranges from about 0.15 to about 0.75 mg/kg/day. In other embodiments, the amount of 6TG administered for in vivo chemoselection ranges from about 0.2 to about 0.7 mg/kg/day. In other embodiments, the amount of 6TG administered for in vivo chemoselection ranges from about 0.25 to about 0.65 mg/kg/day. In other embodiments, the amount of 6TG administered for in vivo chemoselection ranges from about 0.3 to about 0.5 mg/kg/day. In other embodiments, the amount of 6TG administered for in vivo chemoselection ranges from about 0.35 to about 0.55 mg/kg/day. In other embodiments, the amount of 6TG administered for in vivo chemoselection ranges from about 0.4 to about 0.5 mg/kg/day. In other embodiments, the amount of 6TG administered for in vivo chemoselection ranges from about 0.375 mg/kg/day to about 0.425 mg/kg/day. In other embodiments, the amount of 6TG administered for in vivo chemoselection is about 0.35 mg/kg/day. In other embodiments, the amount of 6TG administered for in vivo chemoselection is about 0.375 mg/kg/day. In other embodiments, the amount of 6TG administered for in vivo chemoselection is about 0.4 mg/kg/day. In other embodiments, the amount of 6TG administered for in vivo chemoselection is about 0.425 mg/kg/day. In other embodiments, the amount of 6TG administered for in vivo chemoselection is about 0.45 mg/kg/day. In some embodiments, an amount of 6TG administered per dosage to a patient for in vivo chemoselection is less than 0.8 mg/kg/day. In some embodiments, an amount of 6TG administered per dosage to a patient for in vivo chemoselection is less than 0.75 mg/kg/day. In some embodiments, an amount of 6TG administered per dosage to a patient for in vivo chemoselection is less than 0.7 mg/kg/day. In some embodiments, an amount of 6TG administered per dosage to a patient for in vivo chemoselection is less than 0.65 mg/kg/day. In some embodiments, an amount of 6TG administered per dosage to a patient for in vivo chemoselection is less than 0.6 mg/kg/day. In some embodiments, an amount of 6TG administered per dosage to a patient for in vivo chemoselection is less than 0.55 mg/kg/day. In some embodiments, an amount of 6TG administered per dosage to a patient for in vivo chemoselection is less than 0.5 mg/kg/day.

In some embodiments, a dosage of 6TG is administered to the human patient for in vivo chemoselection one to three times a week on a schedule with a cycle selected from the group consisting of: (i) weekly; (ii) every other week; (iii) one week of therapy followed by two, three or four weeks off; (iv) two weeks of therapy followed by one, two, three or four weeks off; (v) three weeks of therapy followed by one, two, three, four or five weeks off; (vi) four weeks of therapy followed by one, two, three, four or five weeks off; (vii) five weeks of therapy followed by one, two, three, four or five weeks off; and (viii) monthly.

In some embodiments, a dose of 6TG is administered to the human patient every day. In some embodiments, a dose of 6TG is administered to the human patient every other day. In other embodiments, a dose of 6TG is administered to the patient every third day. In some embodiments, a dose of 6TG is administered to the patient every third day for a time period ranging from between about 1 week to about 4 weeks. In other embodiments, a dose of 6TG is administered to the patient every third day for a time period ranging from between about 1 week to about 3 weeks. In some embodiments, a dose of 6TG is administered to the patient every third day for a time period ranging from between about 2 weeks to about 4 weeks.

In some embodiments, in vivo chemoselection comprises providing 1 treatment cycle, i.e. a treatment cycle for selectively depleting cells that express HPRT. In other embodiments, in vivo chemoselection comprises providing between 1 and about 10 treatment cycles. In other embodiments, in vivo chemoselection comprises providing between 1 and about 8 treatment cycles. In other embodiments, in vivo chemoselection comprises providing between 1 and about 6 treatment cycles. In other embodiments, in vivo chemoselection comprises providing between 1 and about 4 treatment cycles. In other embodiments, in vivo chemoselection comprises providing between 1 and about 2 treatment cycles.

In some embodiments, a total number of doses provided in each treatment cycle ranges from between about 1 and about 15 doses. In other embodiments, a total number of doses provided in each treatment cycle ranges from between about 2 and about 15 doses. In other embodiments, a total number of doses provided in each treatment cycle ranges from between about 3 and about 15 doses. In other embodiments, a total number of doses provided in each treatment cycle ranges from between about 3 and about 12 doses. In other embodiments, a total number of doses provided in each treatment cycle ranges from between about 3 and about 10 doses. In other embodiments, a total number of doses provided in each treatment cycle ranges from between about 3 and about 8 doses. In other embodiments, a total number of doses provided in each treatment cycle ranges from between about 3 and about 6 doses. In other embodiments, a total number of doses provided in each treatment cycle ranges from between about 3 and about 5 doses.

In some embodiments, a total amount of 6TG administered per cycle for in vivo chemoselection ranges from between about 1 mg/kg to about 7 mg/kg. In other embodiments, a total amount of 6TG administered per cycle for in vivo chemoselection ranges from between about 1 mg/kg to about 6 mg/kg. In other embodiments, a total amount of 6TG administered per cycle for in vivo chemoselection ranges from between about 1 mg/kg to about 5 mg/kg. In other embodiments, a total amount of 6TG administered per cycle for in vivo chemoselection ranges from between about 1.25 mg/kg to about 5 mg/kg. In other embodiments, a total amount of 6TG administered per cycle for in vivo chemoselection ranges from between about 1.5 mg/kg to about 5 mg/kg. In other embodiments, a total amount of 6TG administered per cycle for in vivo chemoselection ranges from between about 2 mg/kg to about 5 mg/kg. In other embodiments, a total amount of 6TG administered per cycle for in vivo chemoselection ranges from between about 2 mg/kg to about 4.5 mg/kg. In other embodiments, a total amount of 6TG administered per cycle for in vivo chemoselection ranges from between about 2.5 mg/kg to about 4.5 mg/kg. In other embodiments, a total amount of 6TG administered per cycle for in vivo chemoselection ranges from between about 2 mg/kg to about 3 mg/kg. In other embodiments, a total amount of 6TG administered per cycle for in vivo chemoselection ranges from between about 3 mg/kg to about 4 mg/kg.

In some embodiments, the doses of 6TG provided in any single cycle may all comprise the same amounts of 6TG or different amounts of 6TG. In some embodiments, following an initial dose of 6TG having a first amount, at least one subsequent dose of 6TG in the cycle comprises an amount of 6TG which is greater than the first amount. For example, if an initial dose of comprises about 0.4 mg/kg of 6TG, a third dose within the cycle may compromise 0.5 mg/kg. In other embodiments, the first two doses of 6TG in any cycle comprise a first amount of active agent while subsequent doses comprise an increased amount of active agent relative to the first amount. Alternatively, and in other embodiments, the first two doses of 6TG in any cycle comprise a first amount of active agent while subsequent doses comprise a decreased amount of active agent relative to the first amount. In some embodiments, the increased amount is an additional 0.05 mg/kg, 0.075 mg/kg, or 0.1 mg/kg. In some embodiments, half of the doses of 6TG in any cycle may comprise a first amount of 6TG, while the other half of the doses are at an increased amount relative to the first amount. For example, doses 1, 3, and 5 in a cycle may comprise a dose of 0.35 mg/kg, while doses 2 and 4 may comprise an amount of 0.4 mg/kg.

The skilled artisan will also appreciate that the total dose provided in any single cycle may vary from cycle to cycle. For example, a total dose of 6TG in a first cycle may be about 2 mg/kg, but a total dose in a second cycle (or any subsequence) may be 25% more or less than the total dose in the first cycle.

In some embodiments, the amount of 6TG administered per dose is based on a determination of a patient's HPRT enzyme activity. Those of ordinary skill in the art will appreciate that those presenting with higher levels of HPRT enzyme activity may be provided with doses having lower amounts of 6TG. The higher the level of HPRT the greater conversion of 6TG to toxic metabolites. Therefore, the lower dose you would need to administer to achieve the same goal.

Measurement of TPMT genotypes and/or TPMT enzyme activity before instituting 6TG conditioning may identify individuals with low or absent TPMT enzyme activity. As such, in other embodiments, the amount of 6TG administered is based on thiopurine S-methyltransferase (TPMT) levels or TPMT genotype.

In some embodiments, subsequent cycles may comprise the same or different number of doses of 6TG or the same or different amounts of 6TG per dose. For example, a first cycle may comprise five discrete 6TG doses, while a second cycle may comprise six discrete 6TG doses. As another example, each dose in a first cycle may comprise 0.4 mg/kg/day of 6TG, while each dose in a second cycle may comprise 0.45 mg/kg/day of 6TG.

In some embodiments, the dosing with 6TG for in vivo selection follows a stem cell transplant. In some embodiments, the 6TG is dosed about 2 to about 12 weeks after stem cell transplantation. In other embodiments, the 6TG is dosed about 3 to about 8 weeks after stem cell transplantation. In other embodiments, the 6TG is dosed about 2 to about 6 weeks after stem cell transplantation. In other embodiments, the 6TG is dosed about 3 to about 6 weeks after stem cell transplantation.

In some embodiments, dosing with 6TG for in vivo chemoselection takes place at least one day after stem cell transplantation. In other embodiments, dosing with 6TG for in vivo chemoselection takes place from about 1 day to about 90 days after stem cell transplantation. In other embodiments, dosing with 6TG for in vivo chemoselection takes place from about 1 day to about 45 days after stem cell transplantation. In other embodiments, dosing with 6TG for in vivo chemoselection takes place from about 1 day to about 30 days after stem cell transplantation. In other embodiments, dosing with 6TG for in vivo chemoselection takes place from about 1 day to about 20 days after stem cell transplantation. In other embodiments, dosing with 6TG for in vivo chemoselection takes place from about 1 day to about 14 days after stem cell transplantation. In other embodiments, dosing with 6TG for in vivo chemoselection takes place from about 1 day to about 7 days after stem cell transplantation.

### Formulations

In another aspect of the present disclosure are formulations for administration comprising a thiopurine, 6TG, or other purine analog, wherein the active agent is present in an amount ranging from between about 12 mg to about 20 mg.

The formulations of the present disclosure may further comprise one or more pharmaceutically acceptable excipients including, but not limited to, diluents, binders, lubricants, disintegrants, flavoring agents, taste-masking agents, coloring agents, pH modifiers, stabilizers, absorption enhancers, viscosity modifiers, film forming polymers, bulking agents, surfactants, glidants, plasticizers, preservatives, essential oils and sweeteners.

A person skilled in the art will be able to select the suitable excipients or mixtures of excipients for the formulations. In general, the amount of any pharmaceutically acceptable excipient, carrier, and/or additive included within any formulation may vary depending on the desired effect, route of administration, form of the final formulation. In general, however, a total amount of pharmaceutically acceptable excipients, carriers, and/or additives formulated with the formulations may range from about 1% to about 99% by total weight of the formulation. In other embodiments, the total amount of pharmaceutically acceptable excipients, carriers, and/or additives formulated with the formulations may range from about 1% to about 90% by total weight of the composition. In other embodiments, the total amount of pharmaceutically acceptable excipients, carriers, and/or additives formulated with the formulations may range from about 1% to about 80% by total weight of the formulation. In yet other embodiments, the total amount of pharmaceutically acceptable excipients, carriers, and/or additives within the compositions may range from about 1% to about 50% by total weight of the formulation. In other embodiments, the total amount of pharmaceutically acceptable excipients, carriers, and/or additives formulated with the compositions may range from about 5% to about 50% by total weight of the formulation. By way of example only, a formulation may comprise a 50:50 mixture of any of a composition and a pharmaceutically acceptable excipient, carrier, and/or additive.

In some embodiments, a ratio of an amount of 6TG and an amount of a pharmaceutically acceptable excipient or carrier ranges from about 100:1 to about 1:100. In some embodiments, a ratio of an amount of 6TG and an amount of a pharmaceutically acceptable excipient or carrier ranges from about 50:1 to about 1:50. In some embodiments, a ratio of an amount of 6TG and an amount of a pharmaceutically acceptable excipient or carrier ranges from about 25:1 to about 1:25. In some embodiments, a ratio of an amount of a composition and an amount of a pharmaceutically acceptable excipient or carrier ranges from about 10:1 to about 1:10. In some embodiments, a ratio of an amount of a composition and an amount of a pharmaceutically acceptable excipient or carrier ranges from about 5:1 to about 1:5.

Administration to a subject of the formulations according to the present disclosure may be via any common route so long as the target tissue is available via that route. The formulations may conveniently be presented in dosage unit form and may be prepared by any of the methods well known in the art of pharmacy. All methods include the step of bringing the active pharmaceutical ingredient (e.g. a thiopurine, 6TG or another purine analog) into association with an excipient or carrier. In general, the formulations are prepared by uniformly and intimately bringing the active components into association with a liquid carrier or a finely divided solid carrier or both, and then, if necessary, shaping the product into the desired dosage form. In the formulations envisioned herein, the active components are included in an amount sufficient to produce the desired pharmacologic effect.

The formulations may be provided, in general, in the form of discrete units such as hard or soft capsules, tablets, troches or lozenges, each containing a predetermined amount of the active components; in the form of a dispersible powder or granules; in the form of a solution or a suspension in an aqueous liquid or non-aqueous liquid; in the form of syrups or elixirs; or in the form of an oil-in-water emulsion or a water-in-oil emulsion. In such solid dosage forms, the formulations may be mixed with at least one pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For capsules, tablets and pills, the dosage form can also comprise buffering agents.

### EXAMPLE 1

Cycle 1—6TG was administered to a patient two days post-transplantation. The amount of 6TG administered was 0.4 mg/kg. 6TG was then again administered every third day in an amount of 0.4 mg/kg/day for a time period of 13 days. 5 total doses of 6TG were administered and the total amount of 6TG administered was 2 mg/kg. Optional subsequent cycles were spaced two weeks apart.

Optional subsequent cycles-6TG was again administered every third day in an amount of 0.4 mg/kg/day for a time period of 13 days. 5 total doses of 6TG were administered and the total amount of 6TG administered was 2 mg/kg.

### EXAMPLE 2

Two rounds of doses of 6TG were administered for chemoselection following stem cell transplantation.

Round 1 comprised 3 cycles as set forth below:

Cycle 1—6TG was administered to a patient two days post-transplantation. The amount of 6TG administered was 0.4 mg/kg. 6TG was then again administered every third day in an amount of 0.4 mg/kg/day for a time period of 13 days. 5 total doses of 6TG were administered and the total amount of 6TG administered was 2 mg/kg.

Cycle 2—Cycle 2 was initiated 2 weeks after cessation of the first cycle. 6TG was then again administered every third day in an amount of 0.4 mg/kg/day for a time period of 13 days. 5 total doses of 6TG were administered and the total amount of 6TG administered was 2 mg/kg.

Cycle 3—Cycle 2 was initiated 2 weeks after cessation of the second cycle. 6TG was then again administered every third day in an amount of 0.4 mg/kg/day for a time period of 13 days. 5 total doses of 6TG were administered and the total amount of 6TG administered was 2 mg/kg.

Round 2 comprised 3 cycles as set forth below. Rounds 1 and 2 were separated by a period of 2 weeks.

Cycle 4—6TG was administered to a patient two days post-transplantation. The amount of 6TG administered was 0.4 mg/kg. 6TG was then again administered every third day in an amount of 0.4 mg/kg/day for a time period of 13 days. 5 total doses of 6TG were administered and the total amount of 6TG administered was 2 mg/kg.

Cycle 5—Cycle 2 was initiated 2 weeks after cessation of the first cycle. 6TG was then again administered every third day in an amount of 0.4 mg/kg/day for a time period of 13 days. 5 total doses of 6TG were administered and the total amount of 6TG administered was 2 mg/kg.

Cycle 6—Cycle 2 was initiated 2 weeks after cessation of the second cycle. 6TG was then again administered every third day in an amount of 0.4 mg/kg/day for a time period of 13 days. 5 total doses of 6TG were administered and the total amount of 6TG administered was 2 mg/kg.

### EXAMPLE 3

Cycle 1—6TG was administered to a patient two days post-transplantation. The amount of 6TG administered was 0.4 mg/kg. 6TG was then again administered every third day in an amount of 0.3 mg/kg/day for a time period of 19 days. 7 total doses of 6TG were administered and the total amount of 6TG administered was 2.1 mg/kg. Optional subsequent cycles were spaced two weeks apart.

Optional subsequent cycles-6TG was again administered every third day in an amount of 0.3 mg/kg/day for a time period of 19 days. 7 total doses of 6TG were administered and the total amount of 6TG administered was 2.1 mg/kg.

### EXAMPLE 4

Cycle 1—6TG was administered to a patient two days post-transplantation. The amount of 6TG administered was 0.4 mg/kg. 6TG was then again administered every third day in an amount of 0.4 mg/kg/day for a time period of 19 days. 7 total doses of 6TG were administered and the total amount of 6TG administered was 2.8 mg/kg. Optional subsequent cycles were spaced two weeks apart.

Optional subsequent cycles-6TG was again administered every third day in an amount of 0.4 mg/kg/day for a time period of 19 days. 7 total doses of 6TG were administered and the total amount of 6TG administered was 2.8 mg/kg.

### EXAMPLE 5

Cycle 1—6TG was administered to a patient two days post-transplantation. The amount of 6TG administered was 0.4 mg/kg. 6TG was then again administered every third day in an amount of 0.4 mg/kg/day for a time period of 13 days. 5 total doses of 6TG were administered and the total amount of 6TG administered was 2 mg/kg. Optional subsequent cycles were spaced two weeks apart.

Optional subsequent cycles—6TG was again administered every third day in an amount of 0.4 mg/kg/day for a time period of 19 days. 7 total doses of 6TG were administered and the total amount of 6TG administered was 2.8 mg/kg.

### EXAMPLE 6

Cycle 1—6TG was administered to a patient two days post-transplantation. The amount of 6TG administered was 0.4 mg/kg. 6TG was then again administered every third day in an amount of 0.4 mg/kg/day for a time period of 19 days. 7 total doses of 6TG were administered and the total amount of 6TG administered was 2.8 mg/kg. Optional subsequent cycles were spaced two weeks apart.

Optional subsequent cycles—6TG was again administered every third day in an amount of 0.3 mg/kg/day for a time period of 19 days. 7 total doses of 6TG were administered and the total amount of 6TG administered was 2.1 mg/kg.

### EXAMPLE 7

Cycle 1—6TG was administered to a patient two days post-transplantation. The amount of 6TG administered was 0.4 mg/kg. 6TG was then again administered every third day in an amount of 0.3 mg/kg/day for a time period of 19 days. 7 total doses of 6TG were administered and the total amount of 6TG administered was 2.1 mg/kg. Optional subsequent cycles were spaced two weeks apart.

Optional subsequent cycles—6TG was again administered every third day in an amount of 0.4 mg/kg/day for a time period of 13 days. 5 total doses of 6TG were administered and the total amount of 6TG administered was 2 mg/kg.

### EXAMPLE 8

Two rounds of treatment with 6TG were provided to a patient for chemoselection following stem cell transplantation. Following the two rounds of chemoselection, an additional stem cell transplant was carried out. In this way, the second round of chemoselection not only served to increase engraftment of the transplanted cells, but also served to pre-condition the patient for the second stem cell transplant.

Round 1 comprised 3 cycles as set forth below:

Cycle 1—6TG was administered to a patient two days post-transplantation. The amount of 6TG administered was 0.4 mg/kg. 6TG was then again administered every third day in an amount of 0.4 mg/kg/day for a time period of 13 days. 5 total doses of 6TG were administered and the total amount of 6TG administered was 2 mg/kg.

Cycle 2—Cycle 2 was initiated 2 weeks after cessation of the first cycle. 6TG was then again administered every third day in an amount of 0.4 mg/kg/day for a time period of 13 days. 5 total doses of 6TG were administered and the total amount of 6TG administered was 2 mg/kg.

Cycle 3—Cycle 2 was initiated 2 weeks after cessation of the second cycle. 6TG was then again administered every third day in an amount of 0.6 mg/kg/day for a time period of 13 days. 5 total doses of 6TG were administered and the total amount of 6TG administered was 3 mg/kg.

Round 2 comprised 3 cycles as set forth below. Rounds 1 and 2 were separated by a period of 2 weeks.

Cycle 4—6TG was administered to a patient two days post-transplantation. The amount of 6TG administered was 0.4 mg/kg. 6TG was then again administered every third day in an amount of 0.4 mg/kg/day for a time period of 13 days. 5 total doses of 6TG were administered and the total amount of 6TG administered was 2 mg/kg.

Cycle 5—Cycle 2 was initiated 2 weeks after cessation of the first cycle. 6TG was then again administered every third day in an amount of 0.4 mg/kg/day for a time period of 13 days. 5 total doses of 6TG were administered and the total amount of 6TG administered was 2 mg/kg.

Cycle 6—Cycle 2 was initiated 2 weeks after cessation of the second cycle. 6TG was then again administered every third day in an amount of 0.4 mg/kg/day for a time period of 13 days. 5 total doses of 6TG were administered and the total amount of 6TG administered was 2 mg/kg.

Second stem cell transplant—A second stem cell transplantation was carried out two weeks following the end of the sixth cycle. Two days following the second stem cell transplant, two additional rounds of chemoselection with 6TG were initiated.

Round 3 comprised 3 cycles as set forth below:

Cycle 7—6TG was administered to a patient two days post-transplantation. The amount of 6TG administered was 0.4 mg/kg. 6TG was then again administered every third day in an amount of 0.4 mg/kg/day for a time period of 13 days. 5 total doses of 6TG were administered and the total amount of 6TG administered was 2 mg/kg.

Cycle 8—Cycle 2 was initiated 2 weeks after cessation of the first cycle. 6TG was then again administered every third day in an amount of 0.4 mg/kg/day for a time period of 13 days. 5 total doses of 6TG were administered and the total amount of 6TG administered was 2 mg/kg.

Cycle 9—Cycle 2 was initiated 2 weeks after cessation of the second cycle. 6TG was then again administered every third day in an amount of 0.4 mg/kg/day for a time period of 13 days. 5 total doses of 6TG were administered and the total amount of 6TG administered was 2 mg/kg.

Round 4 comprised 3 cycles as set forth below. Rounds 3 and 4 were separated by a period of 2 weeks.

Cycle 10—6TG was administered to a patient two days post-transplantation. The amount of 6TG administered was 0.4 mg/kg. 6TG was then again administered every third day in an amount of 0.4 mg/kg/day for a time period of 13 days. 5 total doses of 6TG were administered and the total amount of 6TG administered was 2 mg/kg.

Cycle 11—Cycle 2 was initiated 2 weeks after cessation of the first cycle. 6TG was then again administered every third day in an amount of 0.4 mg/kg/day for a time period of 13 days. 5 total doses of 6TG were administered and the total amount of 6TG administered was 2 mg/kg.

Cycle 12—Cycle 2 was initiated 2 weeks after cessation of the second cycle. 6TG was then again administered every third day in an amount of 0.4 mg/kg/day for a time period of 13 days. 5 total doses of 6TG were administered and the total amount of 6TG administered was 2 mg/kg.

### Additional Embodiments

1. A method of increasing stem cell engraftment comprising administering to a human patient a dosage of 6TG ranging from between about 0.2 mg/kg/day to about 0.6 mg/kg/day following a stem cell transplant.

2. The method of embodiment 1, wherein the dosage ranges from between about 0.3 mg/kg/day to about 0.5 mg/kg/day.

3. The method of embodiment 2, wherein the dosage is about 0.4 mg/kg/day.

4. The method of embodiment 1, wherein the dosage is administered to the human patient one to three times a week on a schedule with a cycle selected from the group consisting of: (i) weekly; (ii) every other week; (iii) one week of therapy followed by two, three or four weeks off; (iv) two weeks of therapy followed by one, two, three or four weeks off; (v) three weeks of therapy followed by one, two, three, four or five weeks off; (vi) four weeks of therapy followed by one, two, three, four or five weeks off; (vii) five weeks of therapy followed by one, two, three, four or five weeks off; and (viii) monthly.

5. The method of embodiment 1, wherein between about 3 and about 10 dosages of 6TG are administered to the patient over an administration period ranging from 1 week to about 4 weeks.

6. The method of embodiment 5, wherein 4 or 5 dosages of 6TG are administered to the patient over a 14-day period.

7. The method of embodiment 6, wherein the dosages are spaced apart over equal time periods.

8. The method of embodiment 7, wherein subsequent dosages are made every third day.

9. The method of embodiment 6, wherein each dosage comprises the same amount of 6TG.

10. The method of embodiment 6, wherein at least one subsequent dose comprises an amount of 6TG greater than an amount of 6TG in an initial dosage.

11. The method of embodiment 3, wherein the cycle is repeated 4, 6, 8, or 10 times.

12. The method of embodiment 1, wherein a time period between dosing the human patient with 6TG and the stem cell transplant ranges from between about 2 weeks to about 6 weeks.

13. The method of embodiment 5, wherein the time period ranges from between about 3 weeks to about 4 weeks.

14. The method of embodiment 1, wherein subsequent doses of 6TG are separated by a period of at least one day.

15. The method of embodiment 1, wherein an amount of the 6TG administered is based on a measured HPRT-enzyme activity level.

16. The method of embodiment 4, wherein a total amount of 6 TG administered does not exceed 5 mg/kg.

17. A method of conferring myeloprotection comprising: (i) performing a myelosuppressive conditioning step prior to a stem cell transplantation, the myelosuppressive conditioning step comprising administering one or more dosages of 6TG to induce selective myelotoxicity; and (ii) performing a post-transplantation chemoselection step following stem cell transplantation, wherein the post-transplantation conditioning step comprises administering one or more dosages of 6TG, where each of the one or more dosages comprises an amount of 6TG ranging from about 0.3 mg/kg to about 0.5 mg/kg.

18. The method of embodiment 17, wherein the amount of 6TG is about 0.4 mg/kg.

19. The method of embodiment 17, wherein between about 3 and about 10 dosages of 6TG are administered to the patient over an administration period ranging from 1 week to about 4 weeks.

20. The method of embodiment 19, wherein 4 or 5 dosages of 6TG are administered to the patient over a 14-day period.

21. The method of embodiment 20, wherein the dosages are spaced apart over equal time periods.

22. The method of embodiment 21, wherein subsequent dosages are made every third day.

23. The method of embodiment 19, wherein each dosage comprises the same amount of 6TG.

24. The method of embodiment 19, wherein at least one subsequent dose comprises an amount of 6TG greater than an amount of 6TG in an initial dosage.

25. The method of embodiment 19, wherein the cycle is repeated 4, 6, 8, or 10 times.

26. A formulation for oral administration comprising 6TG, wherein the 6TG is present in an amount ranging from between about 12 mg to about 20 mg, and at least one pharmaceutically acceptable excipient.

27. The formulation of embodiment 26, wherein the at least one pharmaceutically acceptable excipient is selected from pregelatinized starch, croscarmellose sodium, povidone, lactose monohydrate, microcrystalline cellulose, and magnesium stearate, and combinations thereof.

28. The formulation of embodiment 26, wherein the formulation comprises an immediate release portion comprising 6TG and an extended release portion comprising 6TG, and wherein the extended release portion permits release of 6TG over a time period ranging from between about 30 minutes to about 12 hours after administration.

29. The formulation of embodiment 28, wherein an amount of the 6TG in the extended release portion ranges from about 50% to about 75% by weight of the extended release portion.

30. The formulation of embodiment 28, wherein the extended release portion comprises (i) a wax, and (ii) a matrix-forming component selected from the group consisting of succinic acid, citric acid, malic acid, stearic acid, succinic acid, lactic acid, aspartic acid, glutamic acid, gluconic acid, acetic acid, formic acid, hydrochloric acid, sulphuric acid, phosphoric acid, hydrophilic polymers, polyethylene glycols, pH dependent acrylate polymers or copolymers, and pore forming agents.

31. A method of increasing stem cell engraftment comprising administering to a human patient a dosage of a purine analog ranging from between about 0.2 mg/kg/day to about 0.6 mg/kg/day following a stem cell transplant.

32. The method of embodiment 31, wherein the dosage ranges from between about 0.3 mg/kg/day to about 0.5 mg/kg/day.

33. The method of embodiment 32, wherein the dosage is about 0.4 mg/kg/day.

34. The method of embodiment 31, wherein the dosage is administered to the human patient one to three times a week on a schedule with a cycle selected from the group consisting of: (i) weekly; (ii) every other week; (iii) one week of therapy followed by two, three or four weeks off; (iv) two weeks of therapy followed by one, two, three or four weeks off; (v) three weeks of therapy followed by one, two, three, four or five weeks off; (vi) four weeks of therapy followed by one, two, three, four or five weeks off; (vii) five weeks of therapy followed by one, two, three, four or five weeks off; and (viii) monthly.

35. The method of embodiment 31, wherein between about 3 and about 10 dosages of a purine analog are administered to the patient over an administration period ranging from 1 week to about 4 weeks.

36. The method of embodiment 35, wherein 4 or 5 dosages of purine analog are administered to the patient over a 14-day period.

37. The method of embodiment 36, wherein the dosages are spaced apart over equal time periods.

38. The method of embodiment 37, wherein subsequent dosages are made every third day.

39. The method of embodiment 31, wherein the purine analog is 6TG.

40. A formulation comprising a purine analog, wherein the purine analog is present in an amount ranging from between about 8 mg to about 240 mg, and at least one pharmaceutically acceptable excipient.

41. The formulation of embodiment 40, wherein the amount is between about 12 mg to about 20 mg.

42. The formulation of embodiment 40, wherein the purine analog is 6TG.

43. The formulation of embodiment 40, wherein the formulation is a solid dosage form.

44. The formulation of embodiment 40, wherein the formulation is a liquid dosage form.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1. A method of increasing an engraftment of genetically modified hematopoietic cells in a human patient following a transplant of the genetically modified hematopoietic cells comprising:
administering to the human patient one or more doses of 6-thioguanine (6TG), wherein an amount of 6TG in each dose of the one or more doses of 6TG ranges from between about 0.2 mg/kg to about 0.6 mg/kg, wherein a total amount of 6TG administered in an administration cycle does not exceed about 7 mg/kg, and wherein a first dose of 6TG is administered to the human patient between about 1 day to about 7 days after the transplant of the genetically modified hematopoietic cells.

2. The method of claim 1, wherein the amount of 6TG in each dose of the one or more doses ranges from between about 0.3 mg/kg to about 0.5 mg/kg.

3. The method of claim 1, wherein the amount of 6TG in each dose of the one or more doses is about 0.4 mg/kg.

4. The method of claim 1, wherein each dose of the one or more doses comprises the same amount of 6TG.

5. The method of claim 1, wherein between about 3 and about 10 doses of 6TG are administered to the human patient during the administration cycle.

6. The method of claim 5, wherein 4 or 5 doses of 6TG are administered to the human patient during the administration cycle.

7. The method of claim 5, wherein the one or more doses of 6TG are spaced apart over equal time periods.

8. The method of claim 5, wherein the one or more doses of 6TG are made every third day.

9. The method of claim 1, wherein the administration cycle has a duration of about four weeks, and wherein the one or more doses of 6TG are made every third day.

10. The method of claim 1, wherein the administration cycle has a duration ranging from between about two weeks to about four weeks, and wherein the one or more doses of 6TG are made every third day.

11. The method of claim 1, wherein the first dose of 6TG is administered 2 days after the transplant of the genetically modified hematopoietic cells.

12. The method of claim 1, wherein the one or more doses of 6G are administered to the human patient over at least two administration cycles

13. The method of claim 1, further comprising measuring an hypoxanthine-guanine phosphoribosyltransferase-enzyme level (HPRT-enzyme level) of the human patient and administering the one or more doses of 6TG based on the measured HPRT-enzyme activity level of the human patient, wherein the amount of 6TG administered in the one or more doses of 6TG is inversely related to the measured HPRT-enzyme activity level.

14. A method of conferring myeloprotection to a human patient comprising: (i) performing a myelosuppressive conditioning step on the human patient prior to a transplantation of genetically modified hematopoietic cells; and (ii) performing a post-transplantation chemoselection step on the human patient following the transplantation of the genetically modified hematopoietic cells, wherein the post-transplantation chemoselection step comprises administering one or more doses of 6-thioguanine (6TG) to the human patient, wherein an amount of 6TG in each dose of the one or more doses of 6TG ranges from between about 0.3 mg/kg to about 0.5 mg/kg, and wherein a first dose of the one or more doses of 6TG is administered 2 days after the transplantation of the genetically modified hematopoietic cells.

15. The method of claim 14, wherein the amount of 6TG in each dose of the one or more doses is about 0.4 mg/kg.

16. The method of claim 14, wherein the post-transplantation chemoselection step occurs in an administration cycle having a duration ranging from about 1 week to about 4 weeks, wherein between about 3 and about 10 doses of 6TG are administered during the administration cycle, and wherein a total dose of 6TG administered during the administration cycle does not exceed about 7 mg/kg.

17. The method of claim 16, wherein the total dose of 6TG administered during the administration cycle does not exceed about 5 mg/kg.

18. The method of claim 16, wherein the total dose of 6TG administered during the administration cycle ranges from between about 2 mg/kg to about 3 mg/kg.

19. The method of claim 14, wherein each dose of the one or more doses comprises the same amount of 6TG.

20. The method of claim 14, wherein the post-transplantation chemoselection step comprises administering the one or more doses of 6TG over at least two administration cycles.
